(19) United States
(12) Plant Patent Application Publication
(10) Pub. No.: US 2003/0106113 P1
(43) Pub. Date: Jun. 5, 2003

(54) Hybrid tea rose plant named 'Frivolous Pink'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/016,068

(22) Filed: Dec. 5, 2001

(51) Int. Cl. A01H 5/00

(52) U.S. Cl. PLT/132

(57) ABSTRACT

A new and distinct Hybrid Tea Rose plant variety with pink flowers.

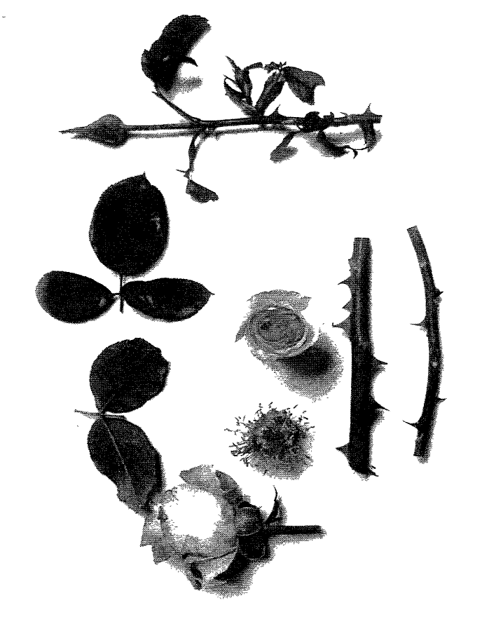

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘FRIVOLOUS PINK’.

### SUMMARY OF THE INVENTION

Among the novel characteristics possessed by this new variety which distinguishes it from other varieties of which I am aware, is its pink flowers.

Asexual reproduction by propagation by budding of the new variety as performed in Wasco, Calif. shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety , with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during the month of November.

### PLANT

Form: Compact

Growth: Medium

Size: Height about 3½ feet; width about 2 feet

Foliage:

Size.—Length about 2 9/16 inches; width about 1⅜ inches.

Quantity.—About 5.

Color.—New foliage: Upper side near 175A; under side near 175C Mature foliage: Upper side near 137A; under side near 137C.

Shape.—Elliptic.

Texture.—Upper side: Smooth Under side: Matte

Edge.—Serrated.

Serration.—Slight.

Petioles.—Top surface color near 143B; Under side near 144B.

Stipules.—Normal.

Rachis.—Few prickles.

Variegation.—None.

Veination.—None.

### WOOD

New shoots:

Bark.—Smooth.

Wood.—Near 46A.

Old wood:

Bark.—Smooth.

Wood.—Near 141C.

Flowering stem: Length about 3 inches

Winter Hardiness: Completely winter hardy

Resistance to Disease: Average

Temperature range: Between 16° and 120° F.

Internode spacing: Usual

Preferred growing conditions: Full sun

Growth habit: Medium vigor

Leaves per stem: About 5

### FLOWER/INFLORESCENCE

Blooming Habit: Recurrent

Bud.—Length about 2 inches; width about 1¼ inches Form: Classic rose Color: Before sepals divide color near 56A; when sepals first divide color near 55B; when petals begin to unfurl color near 55C.

Sepals.—Long, thin on ends Size: Length in relation to bud, about 1 3/16 inches Color: Topside near 145A; bottom near 145B.

Peduncle.—Length: About 2¾ inches Strength: Erect Color: Near 147B.

Flower:

Size.—About 4 inches.

Borne.—Multiple blooms in sprays.

Form.—High centered, opens very flat.

Petalage: About 20

Petaloids in center.—About 3.

Fragrance: Slight

Lasting Quality: About 2 weeks on plant; about 5 days after cutting

Color:

During first 2 days.—Inner petals at flower center topside near 36A; bottom near 36C; outer petals topside near 36B, bottom near 4B.

Base of petals (Point of attachment): Near 4B

General tonality from a distance: Near 36A

Color Change: As bloom ages, fades to near 158C; after 3 or more days, fades to near 158C

Petals:

Texture.—Soft.

Shape.—Wider than long.

Arrangement.—Imbricated.

Appearance.—Velvety.

### REPRODUCTIVE ORGANS

Arrangement of stamens, Regularly arranged around

Filaments and Anthers: styles

Color: Filaments near 4B; Anthers near 4D

Pollen: No pollen

Styles: Medium length

Hips/Seed Vessels/Ovaries

Shape.—Round.

Size.—About ½ inch.

Color.—Near 147B.

Seeds: No seeds

I claim:

1. A new and distinct Hybrid Tea Rose plant of the variety substantially as shown and described.
